(19) United States
(12) Patent Application Publication
(10) Pub. No.: US 2026/0229308 A1
(43) Pub. Date: Aug. 6, 2026

(54) TUMORIGENICITY SCORING FOR CANCER VACCINE DEVELOPMENT AND MEDICAL DECISION MAKING

(71) Applicant: NEC Laboratories America, Inc., Princeton, NJ (US)

(72) Inventors: Jonathan Warrell, Princeton, NJ (US); Renqiang Min, Princeton, NJ (US)

(21) Appl. No.: 19/463,900

(22) Filed: Jan. 29, 2026

Related U.S. Application Data
(60) Provisional application No. 63/751,990, filed on Jan. 31, 2025.

(51) Int. Cl. G16B 20/20; G16B 30/00; G16B 40/20; G16H 20/17

(52) U.S. Cl. CPC G16B 20/20; G16B 30/00; G16B 40/20; G16H 20/17

(57) ABSTRACT

Methods and systems for vaccine generation include determining genome sequence for target. A tumorigenicity score is determined for neoantigens of the target using a variance analysis in a linear random effects model. The neoantigens are ranked based on the tumorigenicity score. A vaccine is generated based on the ranked neoantigens.

### RELATED APPLICATION INFORMATION

This application claims priority to U.S. Patent application No. 63/751,990, filed on Jan. 31, 2025, incorporated herein by reference in its entirety.

### BACKGROUND

### Technical Field

The present invention relates to cancer vaccine development and, more particularly, to identifying tumorigenicity of neoantigens.

### Description of the Related Art

Neoantigen targets may be identified to help develop personalized cancer vaccines and for T-cell receptor engineering. The local context of a genome, such as mutation rate and mutation signature activity, may be analyzed and independent variants that occur in a cancer more frequently than expected may be identified. This results in a small number of variants being identified as drivers, while most variants may be regarded as passengers with neutral effects on the cancer's progression.

### SUMMARY

A method for vaccine generation includes determining genome sequence for target. A tumorigenicity score is determined for neoantigens of the target using a variance analysis in a linear random effects model. The neoantigens are ranked based on the tumorigenicity score. A vaccine is generated based on the ranked neoantigens.

A system for vaccine generation includes a hardware processor and a memory that stores a computer program. When executed by the hardware processor, the computer program causes the hardware processor to determine genome sequence for target, to determine a tumorigenicity score for neoantigens of the target using a variance analysis in a linear random effects model, to rank the neoantigens based on the tumorigenicity score, and to generate a vaccine based on the ranked neoantigens.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram illustrating vaccine development based on tumorigenicity analysis, in accordance with an embodiment of the present invention;

FIG. 2 is a block/flow diagram of a method for determining a tumorigenicity score for neoantigens, in accordance with an embodiment of the present invention;

FIG. 3 is a block/flow diagram of a method for generating and administering a vaccine based on neoantigen tumorigenicity analysis, in accordance with an embodiment of the present invention;

FIG. 4 is a block diagram of a healthcare facility where tailored vaccine development is used to treat cancer, in accordance with an embodiment of the present invention;

FIG. 5 is a block diagram of a computing device that generates and administers cancer vaccines based on tumorigenicity analysis, in accordance with an embodiment of the present invention;

FIG. 6 is a diagram of an exemplary neural network architecture that can be used to implement part of the tumorigenicity analysis, in accordance with an embodiment of the present invention; and

FIG. 7 is a diagram of an exemplary deep neural network architecture that can be used to implement part of the tumorigenicity analysis, in accordance with an embodiment of the present invention.

### DETAILED DESCRIPTION OF PREFERRED EMBODIMENTS

Neoantigen identification for personalized cancer vaccine development may be enhanced by considering the tumorgenicity of a target variant. Tumorigenicity is modeled as a polygenic trait, with a tumorigenicity score being defined to rank neoantigens. Promising shared targets may be identified by analyzing whole genome sequencing (WGS) data from a cohort of patients for a given cancer type, since stably recurring genetic variants in regions of the genome that are translated into proteins result in characteristic protein fragments that may be targeted by the immune system.

The present embodiments identify stably recurring, non-synonymous genetic variants in a given cancer cohort from WGS data that may serve as treatment targets, using tumorigenicity as a factor. For a target to be effective, it should not only be stably recurrent but also exhibit other characteristics such as being presented to the immune system and not duplicating other reference sequences in the human genome. Since WGS data alone cannot provide all the relevant information for identifying idea treatment targets, the target epitopes can be ranked according to their tumorigenicity, which is the propensity of the variants contained in their generating DNA sequences to act as tumor drivers. A target sequence with high tumorigenicity may be expected to stably recur, since its associated variants play a causal role in tumor developments.

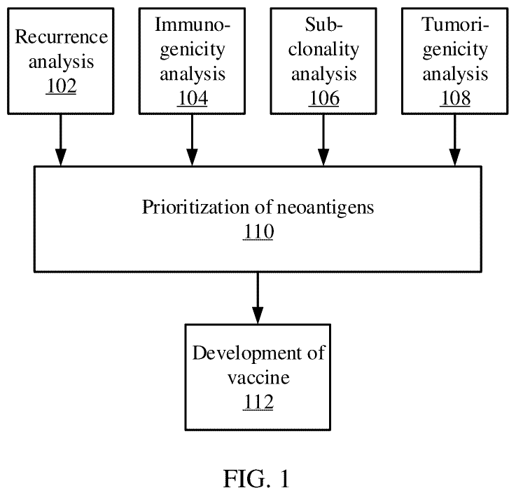

Referring now to FIG. 1, a method for developing a personalized cancer vaccine or engineered T-cells is shown. Multiple different analyses may be used to inform the prioritization of neoantigens, including recurrence analysis 102, immunogenicity analysis 104, subclonality analysis 106, and tumorigenicity analysis 108. Block 110 prioritizes neoantigens at the cohort level or the personal level, incorporating tumorigencitiy information to help identify target neoantigens that are particularly associated with the development of tumors. Block 112 can then develop a new personalized cancer vaccine or engineered T-cell using one or more neoantigens based on a ranked list generated by block 110.

A pool of neoantigens may be drawn from WGS data of tumors from a cohort of N individuals. Given this cancer cohort, block 110 finds stably recurring neoantigens. The somatic variants occurring in a tumor i may be denoted by a binary vector xᵢ, whose j^{th} entry j∈{1, . . . , J} is 1 if the variant occurs in tumor i and is 0 otherwise. For simplicity, consideration may be limited to pointwise non-synonymous mutations such as single nucleotide polymorphisms and pointwise insertions/deletions occurring within exonic regions. Thus only such variants occur in xᵢ.

A reference set of coding sequences may be available, such as the consensus coding sequence (CCDS) dataset. The possible coding sequences may be denoted herein as c₁, . . . , cₙ. Given a fixed neoantigen or epitope length L of interest (e.g., L=9 protein residues), all possible epitope reference coding subsequences of this length may be identified in the reference coding sequence set. For example, all subsequences of length 3L may be identified, since each protein residue corresponds to three DNA bases, starting at multiples of three bases from the start of the sequence. These subsequences may be denoted herein as e₁, . . . , e_{E}, where eₘ∈{A, C, G, T}^{3L}. The notation Eⱼ⊂{1, . . . , E} denotes the set of epitopes affected by variant j, and the notation Jₘ⊂{1, . . . , J} denotes the set of variants affecting epitope eₘ.

The application of a single mutation or multiple mutations to an epitope reference sequence creates a potential target epitope t. These sequences may be indexed using pairs t_{m,S}, where m={1, . . . , E} and S⊆Jₘ. The set of all such target sequences is denoted as T. Block 110 therefore ranks t∈T, ordered by their ability to serve as target epitopes for relevant tumor types. Hence the targets should be stably recurrent (102), presented to the immune system (104), and should not duplicate other reference sequences (106). Tumorigenicity analysis 108 helps to rank target epitopes according to their propensity of their variants to act as tumor drivers.

It is further assumed that a null distribution of non-tumorigenic genotypes is available, represented herein as

$${x}_{i}^{null} ,$$

where i∈{1, . . . , N}. The non-tumorigenic genotypes

$${x}_{i}^{null}$$

are matched to xᵢ in the sense of preserving relevant covariates such as local mutation rate, mutation signature activation, and total mutation burden. The binary term y={0,1} is used to model tumorigenicity, with yᵢ=1 and

$${y}_{i}^{null} = 0.$$

the variants 1, . . . , J can be grouped into functional categories 1, . . . , K, which may be associated with their predicted functional impact on the protein they code for. The set of variants Jₖ belong to category k and k(j) for the category of variant j, with the variant set 1, . . . , J including somatic variants in both observed and null tumor samples.

Tumorigenicity may then be modeled in block 108 using a linear random effects model:

$${y}_{i} = ϕ ( μ + {\sum }_{j} {z}_{ij} {u}_{j} + {ϵ}_{i} )$$

$${u}_{{J}_{k}} ∼ 𝒩 ( 0 , I {σ}_{k}^{2} )$$

$$ϵ ∼ 𝒩 ( 0 , I {σ}_{ϵ}^{2} )$$

where zᵢⱼ is a Z score of the genetic dosage of variant j in individual I, with null samples using indices i=N+{1, . . . , 2N}, μ is a constant offset that is set to 0 when N null samples are used, μⱼ is the random effect of variant j,

$${σ}_{k}^{2}$$

is the additive variance for variants in category k, and φ is the inverse link function.

The choice of φ determines the response model. For example, φ may be chosen to be the identity function (φ(x)=x)) or a probit function (φ(x)=[ψ(x)>t]), where ψ(x) may be the inverse cumulative distribution function of the normal distribution and τ is a threshold value. These examples correspond to the observed and liability scale response models, respectively.

The random effects model for yᵢ may be fitted by maximizing a restricted maximum likelihood (REML) objective, for which the observed scale model takes the form:

$$REML ( {σ}_{1} , ... , {σ}_{K} , σ\_ϵ ) = - \frac{1}{2} log ( \vert \sum ( σ ) \vert ) - \frac{1}{2} {y}^{T} \sum {( σ )}^{- 1} y$$

$$\sum ( σ ) = ZG ( {σ}_{1} , ... , {σ}_{K} ) {Z}^{T} + I {σ}_{ϵ}^{2}$$

$$where G ( {σ}_{1} , ... , {σ}_{K} ) = diag ( {[ {σ}_{k ( j )}^{2} ]}_{j = 1 , ... , J} )$$

and Z is the matrix of Z-scored genetic dosages Zᵢⱼ=zᵢⱼ. The estimated variances \hat{σ}₁, . . . , \hat{σ}_{K}, \hat{σ}_{ϵ} may be used to calculate the best linear unbiased predictor (BLUP) of the variant specific effects:

$$\hat{u} = BLUP ( {\hat{σ}}_{1} , ... , {\hat{σ}}_{K} , {\hat{σ}}_{ϵ} ) = {( {Z}^{T} Z + Λ )}^{- 1} ( {Z}^{T} y )$$

$$Λ = diag ( {[ {σ}_{ϵ}^{2} / {σ}_{k ( j )}^{2} ]}_{j = 1 , ... , J} )$$

This provides a BLUP estimator for y:

$${\hat{y}}_{i} = μ + {\sum }_{j} {z}_{ij} {\hat{u}}_{j}$$

where ŷᵢ denotes the estimator of yᵢ either on the observed or liability scale, depending on the form of φ.

Alternate definitions of tumorigenicity for a target epitope are available. A first alternative uses only the additive variance estimators \hat{σ}₁, . . . , \hat{σ}_{K}, \hat{σ}_{ϵ}, while a second alternative incorporates the BLUP estimator û. In the first case, since the random effects uⱼ are independent, the variance in y may be partitioned for a given reference epitope m as:

$$Var ( y ) = {\sum }_{S ⊆ {J}_{m}} p ( {t}_{m , s} ) {\sum }_{j ∈ {J}_{M}} {z}_{j , [ j ∈ S ]}^{2} {σ}_{k ( j )}^{2} + {\sum }_{j ∈ \{ 1 , ... , J \} \backslash {J}_{m}} {σ}_{k ( j )}^{2} + {\sum }_{{j}_{1} , {j}_{2} ∈ \{ 1 , ... , J \}} Cov ( {z}_{{j}_{1}} , {z}_{{j}_{2}} )$$

where z_{j,b} is the Z-score for variant j for individuals i such that xᵢⱼ=b, and where p(t_{m,S}) is the frequency with which t_{m,S} occurs in the cohort. Given this decomposition, and assuming that the second-order effects are small (e.g., Cov(zⱼ_{₁}, zⱼ_{₂})≈0) for most pairs (j₁,j₂), a tumorigenicity score may be defined based on the additive variance estimators only to be the variance in y in the random effects model accounted for by t_{m,S}.

$${T}_{addVar} ( {t}_{m , S} ) = p ( {t}_{m , S} ) {\sum }_{j ∈ {J}_{m}} {z}_{j , [ j ∈ S ]}^{2} {σ}_{k ( j )}^{2}$$

Alternatively the following decomposition of the variance may be used in the BLUP model:

$$Var ( \hat{y} ) = {\sum }_{j ∈ {J}_{m}} {\hat{u}}_{j}^{2} + {\sum }_{j ∈ \{ 1 , ... , J \} \backslash {J}_{m}} {\hat{u}}_{j}^{2} + {\sum }_{{j}_{1} , {j}_{2} ∈ \{ 1 , ... , J \}} {\hat{u}}_{{j}_{1}} {\hat{u}}_{{j}_{2}} Cov ( {z}_{{j}_{1}} , {z}_{{j}_{2}} )$$

Assuming again that the second-order effects are small, then knowledge that an individual expresses the target epitope t_{m,S} reduces the variance in ŷ by the sum of squares of the BLUP estimates. The BLUP-based tumorigenicity score may be defined as:

$${T}_{BLUP} ( {t}_{m , S} ) = {\sum }_{j ∈ {J}_{m}} {\hat{u}}_{j}^{2}$$

Note that T_{BLUP}(t_{m,S}) is independent of S⊆J_{S}. Hence, for a given reference epitope eₘ, the target may be selected that is most prevalent in the tumor samples, e.g.,

$${S}^{*} = {arg max}_{S} p ( {t}_{m , S} \vert y = 1 ) .$$

The tumorigenicity score may be combined with the other types of analysis to determine vaccine composition via solving an integer programming optimization problem in block 110. There may be N potential neoantigen targets for a patient, with respective tumorigenicity scores T_{1 . . . N}, immunogenicity scores I_{1 . . . N}, and recurrence scores R_{1 . . . N} defined as the fraction of patients having a target somatic mutation in a matched cohort and belonging to subclones 1 . . . M, where Sₘ∈{1 . . . N} represents a set of targets associated with subclone m. Furthermore there may be a budget B<N for how many neoantigens may be included in the vaccine and a threshold C<N for how many neoantigens should target each subclone.

A subset V⊂{1 . . . N} of vaccine neoantigens may be represented by a set of Boolean variables v_{1 . . . N}∈{0,1} such that vₙ=1 iff n∈V. The vaccine composition may be determined by solving an integer linear programming problem:

$${max}_{{v}_{1 … N}} {\sum }_{n} {v}_{n} ( {T}_{n} + α {I}_{n} + β {R}_{n} )$$

$$s . t . {\sum }_{n} {v}_{n} \leq B$$

$${\sum }_{n ∈ {S}_{m}} {v}_{n} \geq C , ∀ m ∈ \{ 1 … M \}$$

where the constants α, β, and C may be set according to experimental data on in vivo systems (such as mouse cancer explant experiments), in vitro systems (such as organoid experiments), in silico systems (such as virtual cell and tissue models), and/or prior clinical data to optimize expected patient response.

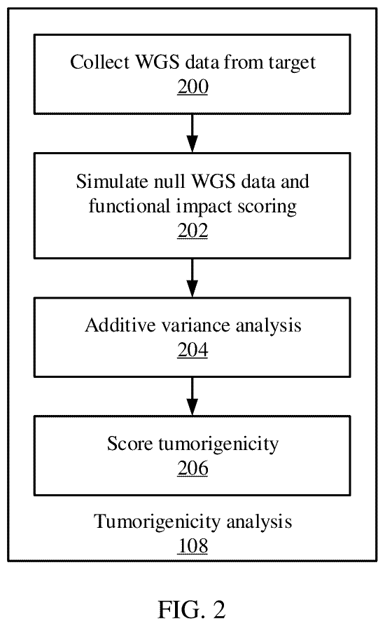

Referring now to FIG. 2, additional detail on the tumorigenicity analysis 108 is shown, following the mathematical approaches described above. WGS data is collected from the target, whether from a target population or a single individual, in block 200. Block 202 simulates null WGS data and functional impact scoring. The simulated samples may be generated by taking somatic mutations from each observed tumor, shuffling the variants so that each original variant is displaced by a distance of not more than, e.g., 500,000 base pairs, to a site with matching tri-nucleotide (or penta-nucleotide) context. This preserves local mutation rates and mutational signatures. Variants may be partitioned into functional categories to provide an estimate of the disruptiveness of each variant on protein function and regulatory effects, where the latter may also be applied to non-coding variants.

Block 204 performs additive variance analysis as described above, for example determining the variance according to additive variance estimators or according to the BLUP model. Based on the variance, block 206 determines a tumorigenicity score.

In some embodiments the tumorigenicity may be determined based on a linear neural network architecture, incorporating biological knowledge of gene expression and gene-variant interactions. The above framework may be extended to incorporate an additional level of training using a neural network model. The linear BLUP model may be replaced by a Large Linear Neural-Network, resulting in the following predictor:

$$\hat{{y}_{ι}} = [ {W}_{L} {b}_{L} ; 01 ] ( [ {W}_{L - 1} {b}_{L - 1} ; 01 ] ( … ( [ {W}_{0} {b}_{0} ; 01 ] [ {z}_{i} ; 1 ] ) ) )$$

where Wₗ, bₗ are respectively the weight matrix and bias vector associated with level l a Linear Neural-Network, and zᵢ is the vector of dosages associated with individual i. The neural network may be trained directly using the cross-entropy between ŷᵢ, and the true label yᵢ. Alternatively, the BLUP estimate may be used as a regularizer. Finally, a prior foundation model which incorporates known biological structure may be used as the initial layers of T_{BLUP}(t_{m,S}), which may be frozen while the other layers are trained. Finally, the effect estimates from the neural network may be calculated as

$$[ \text{?} ; μ ] = [ {W}_{L} {b}_{L} ; 01 ] ( [ {W}_{L - 1} {b}_{L - 1} ; 01 ] ( … ( [ {W}_{0} {b}_{0} ; 01 ] ) ) )$$

which may be substituted for the BLUP-based [symbol] estimates above allowing potential enhanced accuracy through the neural network's architectural bias and the incorporation of prior biological information.

The training losses described above may be summarized as:

$${L}_{1} ( \hat{{y}_{ι}} , {y}_{i} ) = CrossEntropy ( \hat{{y}_{ι}} , {y}_{i} )$$

$${L}_{2} ( \hat{{y}_{ι}} , {y}_{i} ) = CrossEntropy ( \hat{{y}_{ι}} , {y}_{i} ) + α MSE ( \text{?} , \text{?} )$$

$${L}_{3} ( \hat{{y}_{ι}} , {y}_{i} ) = CrossEntropy ( \hat{{y}_{ι}} , {y}_{i} ) + α {L}_{2} ( vec ( {W}_{{l}^{′} … L} , {b}_{{l}^{′} … L} ) )$$

where MSE is the Mean Square Error, and α is a hyperparameter tuned using cross-validation, L₂ is the L₂-loss, vec denotes vectorization, and l′ is the number of layers in the reference foundation model used in the third training scenario above.

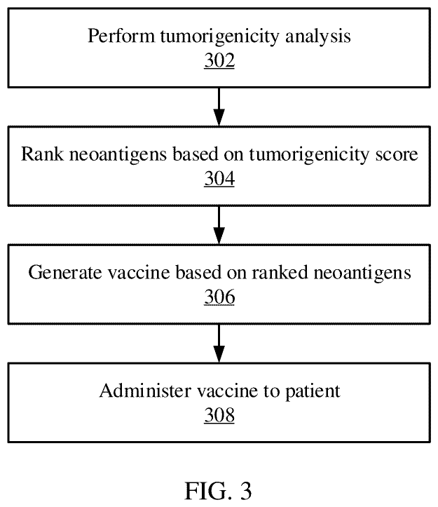

Referring now to FIG. 3, a method of treating a patient with a personalized cancer vaccine is shown. Block 302 performs a tumorigenicity analysis, having sequenced the patient's genome. Block 304 ranks neoantigens based on the tumorigenicity score, with higher-ranked neoantigens being better candidates for a vaccine. Block 306 then generates the vaccine based on the ranked neoantigens, for example producing one or more of the highest-ranked neoantigens. Block 308 then administers the vaccine to the patient to treat the cancer.

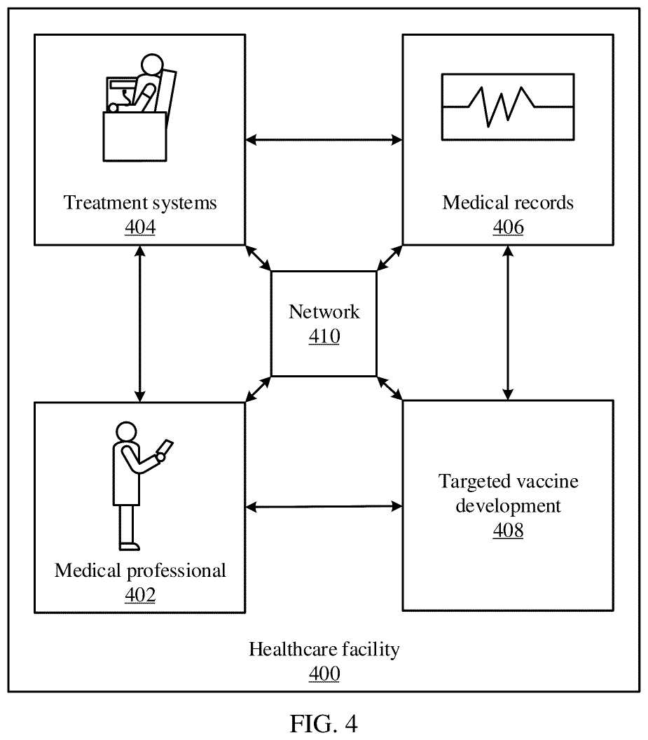

Referring now to FIG. 4, a diagram of time series analysis is shown in the context of a healthcare facility 400. Targeted vaccine development 408 may be used to create a vaccine that is tailored to a patient's specific cancer, for example based on test results and medical records 406. Information regarding tumorigenicity may be used to assist medical professionals 402 in medical decision making.

The healthcare facility may include one or more medical professionals 402 who review information extracted from a patient's medical records 406 to determine their healthcare and treatment needs. These medical records 406 may include self-reported information from the patient, test results, and notes by healthcare personnel made to the patient's file. Treatment systems 404 may furthermore monitor patient status to generate medical records 406 and may be designed to automatically administer and adjust treatments as needed.

The targeted vaccine development 408 may be used to identify neoantigens that are tailored to target variants with high tumorigenicity. This improves the efficacy of the vaccine that is developed based on the selected neoantigens. For example a tissue sample may be taken from a patient and may be part of the medical records 406.

The different elements of the healthcare facility 400 may communicate with one another via a network 410, for example using any appropriate wired or wireless communications protocol and medium. Thus targeted vaccine development 408 receives data from treatment systems 404, medical professionals 402, and from medical records 406, and generates a vaccine that is targeted to a patient's specific cancer. The targeted vaccine development 408 may further coordinate with treatment systems 404 in some cases to automatically administer or alter a treatment. For example, the targeted vaccine development 408 may be used to determine a course of treatment and automatically administering the vaccine via the treatment systems 404.

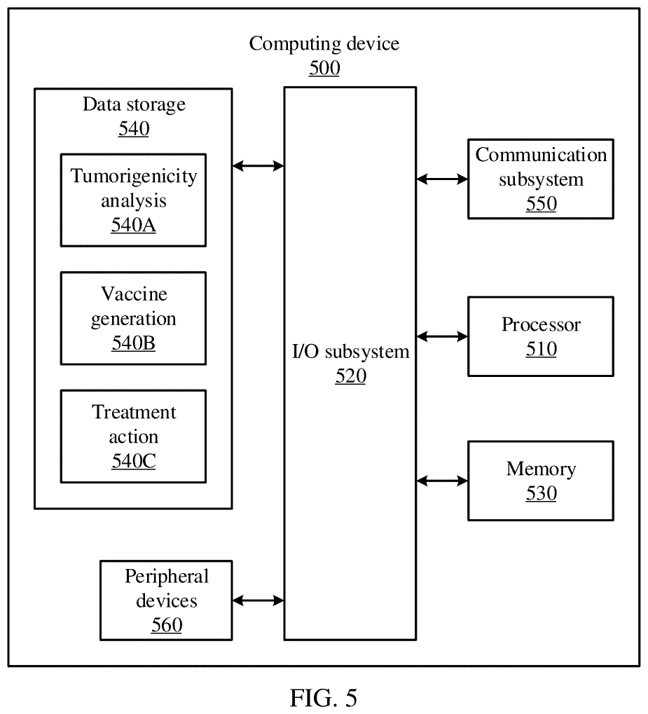

Referring now to FIG. 5, an exemplary computing device 500 is shown, in accordance with an embodiment of the present invention. The computing device 500 is configured to perform targeted vaccine development.

The computing device 500 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 500 may be embodied as one or more compute sleds, memory sleds, or other racks, sleds, computing chassis, or other components of a physically disaggregated computing device.

As shown in FIG. 5, the computing device 500 illustratively includes the processor 510, an input/output subsystem 520, a memory 530, a data storage device 540, and a communication subsystem 550, and/or other components and devices commonly found in a server or similar computing device. The computing device 500 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 530, or portions thereof, may be incorporated in the processor 510 in some embodiments.

The processor 510 may be embodied as any type of processor capable of performing the functions described herein. The processor 510 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 530 may be embodied as any type of volatile or non-volatile memory or data storage capable of performing the functions described herein. In operation, the memory 530 may store various data and software used during operation of the computing device 500, such as operating systems, applications, programs, libraries, and drivers. The memory 530 is communicatively coupled to the processor 510 via the I/O subsystem 520, which may be embodied as circuitry and/or components to facilitate input/output operations with the processor 510, the memory 530, and other components of the computing device 500. For example, the I/O subsystem 520 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 520 may form a portion of a system-on-a-chip (SOC) and be incorporated, along with the processor 510, the memory 530, and other components of the computing device 500, on a single integrated circuit chip.

The data storage device 540 may be embodied as any type of device or devices configured for short-term or long-term storage of data such as, for example, memory devices and circuits, memory cards, hard disk drives, solid state drives, or other data storage devices. The data storage device 540 can store program code 540A for tumorigenicity analysis, 540B for vaccine generation, and/or 540C for performing treatment actions. Any or all of these program code blocks may be included in a given computing system. The communication subsystem 550 of the computing device 500 may be embodied as any network interface controller or other communication circuit, device, or collection thereof, capable of enabling communications between the computing device 500 and other remote devices over a network. The communication subsystem 550 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 500 may also include one or more peripheral devices 560. The peripheral devices 560 may include any number of additional input/output devices, interface devices, and/or other peripheral devices. For example, in some embodiments, the peripheral devices 560 may include a display, touch screen, graphics circuitry, keyboard, mouse, speaker system, microphone, network interface, and/or other input/output devices, interface devices, and/or peripheral devices.

Of course, the computing device 500 may also include other elements (not shown), as readily contemplated by one of skill in the art, as well as omit certain elements. For example, various other sensors, input devices, and/or output devices can be included in computing device 500, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 500 are readily contemplated by one of ordinary skill in the art given the teachings of the present invention provided herein.

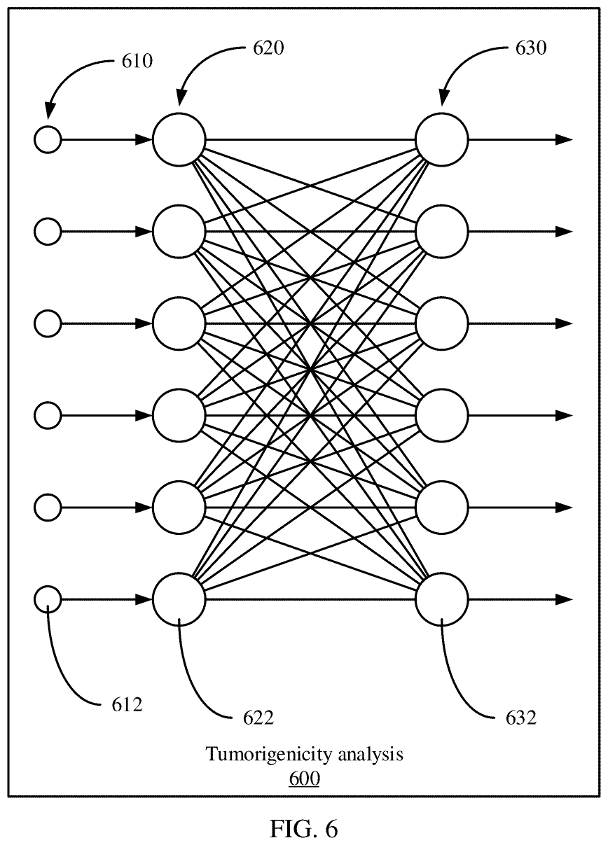

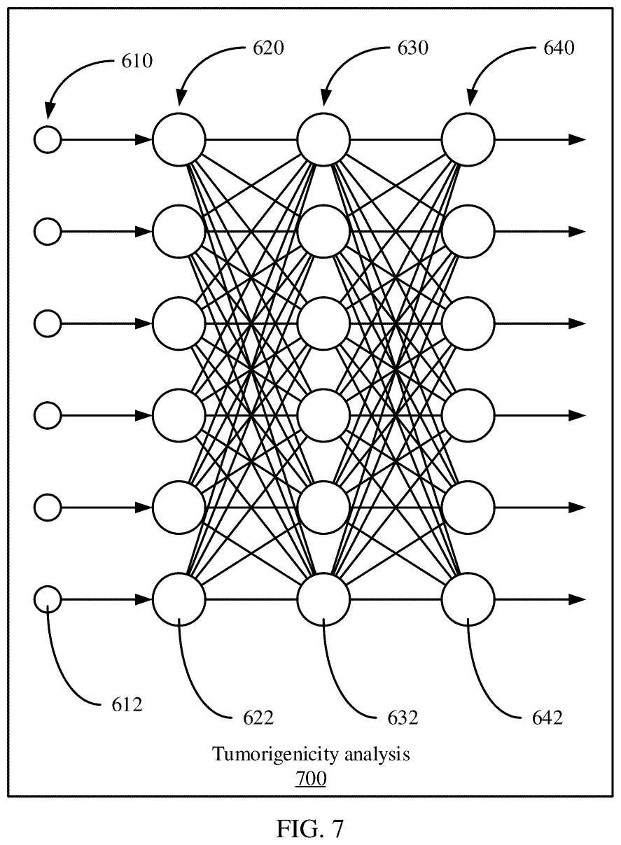

Referring now to FIGS. 6 and 7, exemplary neural network architectures are shown, which may be used to implement parts of the present machine learning models, such as in tumorigenicity analysis 600/700. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 620 of source nodes 622, and a single computation layer 630 having one or more computation nodes 632 that also act as output nodes, where there is a single computation node 632 for each possible category into which the input example could be classified. An input layer 620 can have a number of source nodes 622 equal to the number of data values 612 in the input data 610. The data values 612 in the input data 610 can be represented as a column vector. Each computation node 632 in the computation layer 630 generates a linear combination of weighted values from the input data 610 fed into input nodes 620, and applies a non-linear activation function that is differentiable to the sum. The exemplary simple neural network can perform classification on linearly separable examples (e.g., patterns).

A deep neural network, such as a multilayer perceptron, can have an input layer 620 of source nodes 622, one or more computation layer(s) 630 having one or more computation nodes 632, and an output layer 640, where there is a single output node 642 for each possible category into which the input example could be classified. An input layer 620 can have a number of source nodes 622 equal to the number of data values 612 in the input data 610. The computation nodes 632 in the computation layer(s) 630 can also be referred to as hidden layers, because they are between the source nodes 622 and output node(s) 642 and are not directly observed. Each node 632, 642 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 632 in the one or more computation (hidden) layer(s) 630 perform a nonlinear transformation on the input data 612 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for vaccine generation, comprising:
determining genome sequence for target;
determining a tumorigenicity score for a plurality of neoantigens of the target using a variance analysis in a linear random effects model;
ranking the plurality of neoantigens based on the tumorigenicity score; and
generating a vaccine based on the ranked neoantigens.

2. The method of claim 1, wherein determining the tumorigenicity score includes determining a variance based on additive variance estimators.

3. The method of claim 2, wherein the tumorigenicity score for a neoantigen sequence t_{m,S} is calculated as:
$${T}_{addVar} ( {t}_{m , S} ) = p ( {t}_{m , S} ) {\sum }_{j ∈ {J}_{m}} {z}_{j , [ j ∈ S ]}^{2} {σ}_{k ( j )}^{2}$$
where p(t_{m,S}) is a frequency of the neoantigen, Jₘ is a set of variants of the neoantigen,
$${z}_{j , [ j ∈ S ]}^{2}$$
is a Z-score for variant j among a subset of variants, and
$${σ}_{k ( j )}^{2}$$
is an additive variance for variance in category k(j).

4. The method of claim 1, wherein determining the tumorigenicity score includes determining a variance based on best linear unbiased predictor (BLUP) estimator.

5. The method of claim 4, wherein the tumorigenicity score for a neoantigen sequence t_{m,S} is calculated as:
$${T}_{BLUP} ( {t}_{m , S} ) = {\sum }_{j ∈ {J}_{m}} {\hat{u}}_{j}^{2}$$
where Jₘ is a set of variants of the neoantigen and
$${\hat{u}}_{j}^{2}$$
is an element or the BLUP estimator.

6. The method of claim 1, wherein the linear random effects model includes an inverse link function that is implemented as a probit function using an inverse of a cumulative distribution function.

7. The method of claim 1, further comprising simulating a null genome sequence, wherein determining the tumorigenicity score preserves covariates with the null genome sequence.

8. The method of claim 1, wherein determining the tumorigenicity score uses a machine learning model trained on biological knowledge of gene expression and gene-variant interactions.

9. The method of claim 1, wherein the tumorigenicity score is used to assist in medical decision making.

10. The method of claim 1, further comprising administering the vaccine to a patient, wherein the target is a tissue sample taken from a cancer of the patient.

11. A system for vaccine generation, comprising:
a hardware processor; and
a memory that stores a computer program which, when executed by the hardware processor, causes the hardware processor to:
determine genome sequence for target;
determine a tumorigenicity score for a plurality of neoantigens of the target using a variance analysis in a linear random effects model;
rank the plurality of neoantigens based on the tumorigenicity score; and
generate a vaccine based on the ranked neoantigens.

12. The system of claim 11, wherein the computer program causes the hardware processor to determine a variance based on additive variance estimators as part of the determination of tumorigenicity score.

13. The system of claim 12, wherein the tumorigenicity score for a neoantigen sequence t_{m,S} is calculated as:
$${T}_{addVar} ( {t}_{m , S} ) = p ( {t}_{m , S} ) {\sum }_{j ∈ {J}_{m}} {z}_{j , [ j ∈ S ]}^{2} {σ}_{k ( j )}^{2}$$
where p(t_{m,S}) is a frequency of the neoantigen, Jₘ is a set of variants of the neoantigen,
$${z}_{j , [ j ∈ S ]}^{2}$$
is a Z-score for variant j among a subset of variants, and
$${σ}_{k ( j )}^{2}$$
is an additive variance for variance in category k(j).

14. The system of claim 11, wherein the computer program further causes the hardware processor to determine a variance based on best linear unbiased predictor (BLUP) estimator as part of the determination of the tumorigenicity score.

15. The system of claim 14, wherein the tumorigenicity score for a neoantigen sequence t_{m,S} is calculated as:
$${T}_{BLUP} ( {t}_{m , S} ) = {\sum }_{j ∈ {J}_{m}} {\hat{u}}_{j}^{2}$$
where Jₘ is a set of variants of the neoantigen and
$${\hat{u}}_{j}^{2}$$
is an element of the BLUP estimator.

16. The system of claim 11, wherein the linear random effects model includes an inverse link function that is implemented as a probit function using an inverse of a cumulative distribution function.

17. The system of claim 11, wherein the computer program further causes the hardware processor to simulate a null genome sequence, wherein determining the tumorigenicity score preserves covariates with the null genome sequence.

18. The system of claim 11, wherein determination of the tumorigenicity score uses a machine learning model trained on biological knowledge of gene expression and gene-variant interactions.

19. The system of claim 11, wherein the tumorigenicity score is used to assist in medical decision making.

20. The system of claim 11, wherein the computer program further causes the hardware processor to administer the vaccine to a patient, wherein the target is a tissue sample taken from a cancer of the patient.
